(19) United States
(12) Patent Application Publication
(10) Pub. No.: US 2005/0189252 A1
(43) Pub. Date: Sep. 1, 2005

(54) Sterilization container

(76) Inventors: Jennifer Radovich Naylor, Batavia, IL (US); Cheryl Marie Miller, Evanston, IL (US)

Correspondence Address: WELSH & KATZ, LTD, 120 S RIVERSIDE PLAZA, 22ND FLOOR, CHICAGO, IL, 60606, US

(21) Appl. No.: 11/058,862

(22) Filed: Feb. 16, 2005

Related U.S. Application Data
(60) Provisional application No. 60/545,782, filed on Feb. 19, 2004.

(51) Int. Cl. A61B 19/02

(52) U.S. Cl. 206/439; 206/363; 206/370; 422/292

(57) ABSTRACT

A sterilization pouch incorporates a selected bottom paper layer to which is bonded to a transparent resin top layer. The paper layer carries a printed self-sealed tape which is covered by a removable sterilization indicating strip. The pouch can be formed with two elongated spaced apart side seals which terminate in an end seal which is straight or chevron-shaped. Various peel designs can be incorporated.

### CROSS REFERENCE TO RELATED APPLICATION

This application claims the benefit of the filing date of U.S. Provisional Application No. 60/545,782 filed on Feb. 19, 2004 and entitled “Sterilization Container”.

### FIELD OF THE INVENTION

The invention pertains to devices usable to facilitate the sterilization and/or organization of medical or dental instruments. More particularly, the invention pertains to flexible, sealable sterilization containers or pouches which also carry visual indicia of a prior sterilization process.

### BACKGROUND OF THE INVENTION

Medical and dental personnel regularly need to organize and sterilize pre-selected groups of instruments that are used in carrying out one procedure or another. Sterilization, as is well known, is essential for all instruments, particularly those which are reusable instruments. Disposable instruments can be sterilized at manufacture and then sealed in a sterile state until used. Such instruments are then appropriately disposed of.

Reusable instruments on the other hand, not only need to be repeatedly field sterilizable, they also need to be groupable, based on a procedural basis, to improve the cost effective delivery of the associated medical or dental procedure. The benefits of being able to group and then sterilize instruments for repeated use are well known.

One known approach to grouping and sterilizing instruments involves the use of sterilizable metal or resin containers or cassettes. Such cassettes can be loaded with a variety of either new or used instruments that are associated with a selected procedure. The cassette, along with the instruments, can then be sterilized and stored at a sterile state until ready for use.

The loaded prestored cassettes can be made available to the medical or dental professional when the respective procedure is to be carried out. Such cassettes also faciliate the color coding of groups of instruments in accordance with the preference of one professional versus another.

Some known cassettes have been disclosed in U.S. Pat. Nos. 4,541,992, 4,854,475, 5,215,726, 5,294,413, and 5,433,929 all of which have been assigned to the assignee hereof. The disclosures of the noted patents are hereby incorporated by reference.

Another approach to organization and sterilization incorporates sterilizable resin pouches. Such pouches represent a less expensive alternate to the use of either metal or resin cassettes. However, durability and stability of such pouches, both before and after sterilization, and during storage, continue to present ongoing issues.

It would be desirable to be able to combine the beneficial aspects of metal or resin cassettes along with those of resin pouches. In addition, it would be desirable to be able to upgrade the convenience and ease of use of such pouches with or without associated cassettes.

### BRIEF DESCRIPTION OF THE DRAWINGS

FIG. 1 is an isometric view of a medical or dental pouch in accordance with the invention;

FIG. 1A is a sectional view taken along plane 1A-1A as in FIG. 1;

FIG. 2 is a view of an alternate pouch which has been filled with a cassette which carries a plurality of dental instruments associated with a particular procedure;

FIG. 3 is a view of another alternate in accordance with the invention;

FIG. 4 is a view of yet another alternate in accordance with the invention;

FIG. 5 is a sectional view taken along plane 5-5 of FIG. 4; and

FIGS. 6A-C illustrate aspects of using pouches in accordance with the invention.

### DETAILED DESCRIPTION OF THE INVENTION

While embodiments of this invention can take many different forms, specific embodiments thereof are shown in the drawings and will be described herein in detail with the understanding that the present disclosure is to be considered as an exemplification of the principles of the invention, as well as the best mode of practicing same, and is not intended to limit the invention to the specific embodiment illustrated.

Embodiments of sterilization containers or pouches in accordance with the invention have been designed to accommodate cassettes as well as other types of dental equipment, and/or instruments. The embodiments can be implemented in several different sizes. For example, multiple sizes can be provided to take into account different sizes of cassettes or other products, such as individual instruments, parts or small cassettes/boxes, all without limitation. Additional sizes can be added to take into account additional cassette designs, and customer needs all without departing from the spirit and scope of the invention.

The disclosed sterilization containers will cost effectively increase office efficiencies. As described below, the cassettes, or other items to be sterilized need only be inserted into a respective one of the containers. The container can be sealed and then, for example, sterilized using steam or chemical vapors. It will be understood that the form of sterilization is not a limitation of the invention.

Disclosed embodiments of pouches incorporate preferably 37-45 pound autoclavable paper as a bottom layer. This layer will provide added strength reducing the chance of product (cassette) puncturing the container. The paper layer can be printed with branding information and a steam indicator. It will be understood, that alternates include a multi-layered structure of a selected paper and layer(s) of an appropriate resin, for example, polypropylene. In another alternate, multiple layers of an autoclavable resin could be combined with a paper layer that only in part covers the bottom.

A top layer, for example, preferably at 1.5-2 mil transparent polypropylene, could be used to close the top of the pouch. The top and bottom layers, the polypropylene and the paper can then be heat sealed together. Preferably, at least a quarter inch seal width will be provided on three sides of the pouch. Other types of sealing, such as ultrasonic welding could also be used.

The end can be formed chevron style. The edges could also be formed with expansion elements, such as gussets, so the pouch can be expandable if desired.

At an open end of the pouch, where the product such as instruments or a cassette is to be inserted, a self-seal strip can be provided that will preferably be printed with a steam indicator. Different sizes can be used. For example, this strip could be on the order of ½-¾ to one inch wide and extend across the entire end of the container. Shorter and/or narrower strips could also be used.

In one disclosed embodiment, the indicator strip is designed to be peeled off and placed in the container with the product before it is sealed and sterilized. The purpose for the indicator strip is to provide an additional indicator that the pouch has been properly sterilized based on temperature and steam.

A fold line can be provided adjacent to the indicator tape. The container will thus be pre-folded to make closure easier for the end-user. The other end of the container can include a squared off bottom with a corner peel on one side allowing for easy opening of the pouch after processing. Theses corners can also have corner tack seals to prevent premature opening.

An alternate embodiment can preferably include a built in indicator strip and a one-half to one inch self-seal closure. The far end of the pouch can have a chevron shape. Such pouches can be used to sterilize individual instruments, parts or small cassettes/boxes for example.

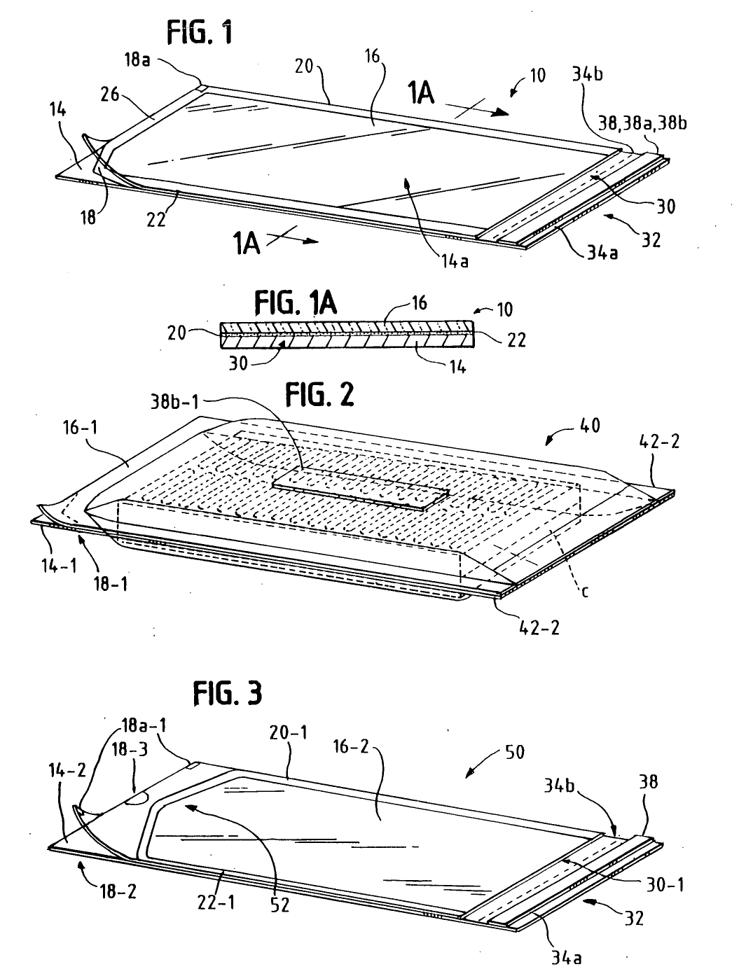

FIG. 1 illustrates a sterilization pouch 10 which is formed of an elongated multi-layer composite of an autoclavable paper 14, a lower layer, and a transparent resin 16 as an upper or top layer. The paper layer 14 and resin layer 16 are sealed along elongated side edges with seals 20, 22. Heat sealing or ultrasonic welding can be used all without limitation to form such seals. Joining the sealed edges 20, 22 is an end seal 26 which could be linear, as illustrated in FIG. 1 or chevron-shaped, best seen in FIG. 3.

The three sealed edges 20, 22 and 26 form an open interior region between the paper layer 14 and resin layer 16. The open interior region, indicated generally at 30, can be filled with various types of instruments. Alternately, the instruments can first be loaded in a cassette and the cassette inserted into the container 10.

Container 10 is closed at a proximal end indicated generally at 32 by a flap 34a which has free end. Flap 34a is bounded by a preformed fold line 34b. The flap 34a carries a two-part self-seal strip 38. The strip 38 includes a preprinted self-seal elongated adhesive layer 38a which is covered by a removable sterilization indicator 38b.

When the container 10 has been filled with the appropriate instrument(s) or cassette, the sterilization indicator 38b is removed and inserted into the region 30. The pouch 10 is then sealed with the tape 38a by folding flap 34a along line 34b. During the sterilization process, the indicator 38b changes color providing a permanent indicium that the sterilization process has taken place. When the paper layer 14 carries a pre-printed (or otherwise affixed) sterilization indicator, indicated generally at 14a, that indicator will also change color as a result of a sterilization process.

Subsequently, when the instruments are to be used, the pouch 10 can easily be opened by separating the layers 14, 16 at a peelable corner 18. It will be understood that the exact location and shape of the peelable geometry is not a limitation of the present invention. Multiple peelable geometries can be provided. For example, one can be provided in each corner. Alternately, peelable geometries can be provided midway along the edges 20, 22 or end 26. For extra security, corner tack seals 18a (to prevent premature separation) can be provided.

FIG. 1A is a sectional view taken along plane 1A-1A of FIG. 1 Top layer 16 is illustrated bonded to lower layer 14 at seals 20, 22.

FIG. 2 is a view of an alternate form of a container 40. The container 40 includes a base paper layer 14-1 and a resin top layer 16-1 comparable to the layers 14, 16 discussed above. In the pouch 40, the layers 14-1, 16-1 can be bonded together along side edges by seals 42-1, -2 with either a straight seam illustrated in FIG. 1, seals 20, 22 or with expansion elements such as gussets. Such expansion elements make insertion of the instruments or a cassette C, as illustrated in FIG. 2 in phantom, convenient.

As illustrated in FIG. 2, the pouch 40 has been sealed with tape comparable to the tape 38a. An indicator strip 38b-1, removed from the sealing tape, has been placed in the container 40 along with the cassette C. As a result of sterilization, the indicator strip 38b-1 changes color and provides a permanent indicium that the sterilization process has been completed.

FIG. 3 illustrates another form of a container 50 in accordance with the invention. The container 50 is formed of a lower paper layer 14-2 which can also carry a preprinted steam or chemcial sterilization indicator. The paper layer 14-2 is covered, as discussed above, with a transparent resin layer 16-2. Side edges can be heat sealed or ultrasonically welded together 20-1, 22-1. Alternately, gussets such as illustrated adjacent to seals 42-1, 42-2 can be provided (as shown in FIG. 2). The container 50 exhibits a chevron-shaped closed end seal 52. The seals 20-1, 22-1 and 50 together form an open region 30-1. The region 30-1 can be closed and sealed by flap 34a as discussed above.

Corner peel geometry 18-2 as well as mid-edge peel geometry 18-3 can be provided for convenient opening of the pouch 50. Safety corner tack seals 18a-1 can be included.

The pouch 50 can be used with instruments that have been directly inserted into the internal region 30-1. Alternately, a cassette such as the cassette C can be filled with instruments and then inserted into the region 30-1 prior to the pouch 50 being sealed and autoclaved.

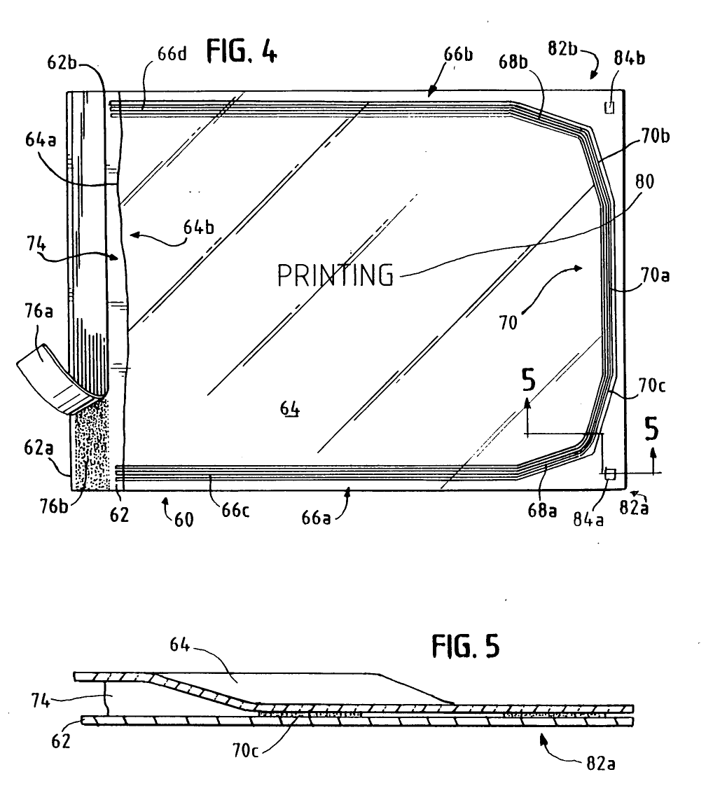

FIGS. 4, 5 illustrate another embodiment of a container 60 in accordance with the invention. The container 60 includes for example a base paper layer 62 and a resin, perhaps transparent, over layer 64.

The base layer 62 and resin layer 64 are bonded together along first and second sides by elongated linear regions 66a, b. The bonding regions 66a, b can be formed using heat sealing or ultrasonic welding or adhesive or any other method which will provide a sterilization safe seal between base layer 62 and over layer 64.

Each of the sealing regions 66a, b terminates in the ends 68a, b. The ends 68a, b are oriented at an angle in a range on the order of 2-5 degrees relative to the regions 66a, b.

The sealing or bonding regions 66a, b are joined by an end sealing or bonding region 70. The region 70 can include an elongated, generally linear central bonding section 70a. The section 70a has first and second end bonding regions 70b, c, which extend at an angle to the elongated section 70a. The angle can be in the range of 2-5°. The end sections 70b, c sealably intersect sealed regions 68a, b thereby forming a closed three sided seal between the base layer 62 and over layer 64.

Side regions 66a, b also terminate at ends 66c, d at an open, unsealed, end 64a of over layer 64. The base layer 62 extends to a flap 62a beyond an end 64a of the cover layer 64. The flap 62a carries a peel-off strip 76a which is releasably bonded to the flap 62a by an adhesive layer 76b.

An internal region 74 of the pouch 60 can be filled with instruments, or alternately with a cassette C that contains instruments, medical or dental devices or the like. The strip 76a can be peeled off of the adhesive 76b. The flap 62a can be rotated about a fold line 62b which will bring the adhesive layer 76b into contact with an end section 64b of the over layer 64. The adhesive 76b will adhere to the end section 64b forming the fourth side of a seal thereby completely closing the internal region 74.

Prior to the closure process, the peel-off strip 76a which can carry a preprinted sterilization indicator, or, is formed of a sterilization indicating material, can be inserted into the region 74. As a result of a sterilization process, which could be for example, steam sterilization or chemical vapor sterilization, the peel-off strip 76a or the indicium on the peel-off strip 76a changes color indicating that the sterilization process has taken place. Additionally, the base layer 62 can be printed with a sterilization indicator 80 of an arbitrary shape. The indicator 80 changes color, for example, from a white to a brown, as a result of steam sterilization or chemical vapor sterilization.

The pouch 60 can also carry peelable regions indicated generally at 82a, b where the over layer 64 can readily be peeled off the base layer 62 after the sterilization process or when the sterilized contents are to be accessed. In this regard, the base layer 62 can be bonded to over layer 64 at tack points 84a, 84b to minimize the likelihood of the container or pouch 60 opening pre-maturely.

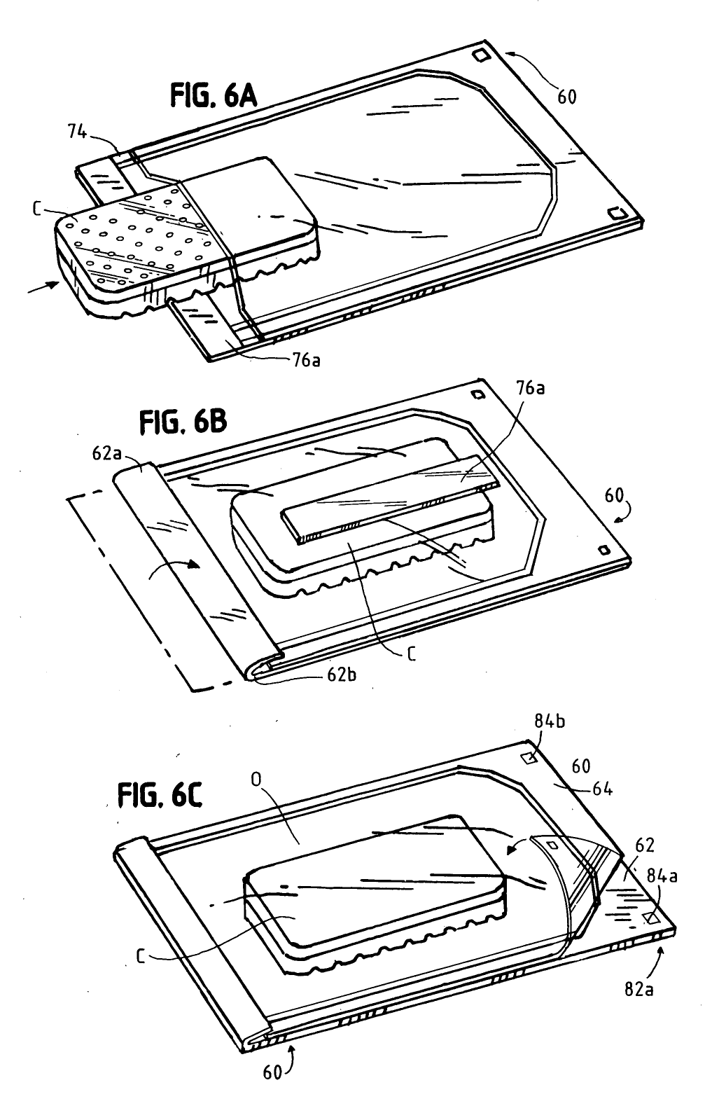

FIGS. 6A, B and C illustrate aspects of usage of containers which embody the present invention such as the container 60. As illustrated in FIG. 6A, an instrument filled cassette C can be inserted into the internal region 74 of the container 60. As illustrated in FIG. 6B, subsequent to the cassette C being inserted into the region 74, the tear-off strip 76a can be removed from flap 62a and inserted into region 74. Flap 61a can then be folded along fold lines 62b so that adhesive layer 76b adheres to an end section 64b thereby sealing pouch 60 closed.

Subsequent to sterilization, the over layer 64 can be separated from the base layer 62 at one or both of the tack regions 84a, b and pulled in a direction generally indicated at O to separate layers 62, 64 for purposes of removing cassette C from the pouch 60 to use the instruments therein. It will be understood that the peel regions associated with tack points 84a, b can assume a variety of shapes and without departing from the spirit and scope of the present invention.

It will be understood that while pouches having paper/resin layers have been discussed above, alternate combinations are possible. For example paper/paper, resin/paper or resin/resin layers can be combined using various sealing processes. All such combinations come within the spirit and scope of the invention.

While embodiments of this invention can take many different forms, specific embodiments thereof are shown in the drawings and will be described herein in detail with the understanding that the present disclosure is to be considered as an exemplification of the principles of the invention, as well as the best mode of practicing same, and is not intended to limit the invention to the specific embodiment illustrated.

1. A flexible container comprising:
a first planar layer bonded to a second planar layer so as to form a partly bounded receiving region, one of the layers is, at least in part, transparent, and, a sterilization indicium, the indicium is observable through the transparent, at least in part, layer.

2. A container as in claim 1 where the layers are flexible.

3. A container as in claim 1 where one of the layers comprises one of resin or paper.

4. A container as in claim 3 where the indicium is printed on one of the layers.

5. A container as in claim 1 where the indicium is carried on one of the layers.

6. A container as in claim 5 where the indicium is printed on one of the layers.

7. A container as in claim 6 where one of the layers comprises one of resin or paper.

8. A container as in claim 7 where one of the layers carries an integrally formed closure flap.

9. A container as in claim 8 where the flap carries a self-adhesive layer.

10. A container as in claim 9 where the flap carries the indicium.

11. A container as in claim 10 where the indicium is bounded, in part, by a fold line.

12. A container as in claim 11 where the indicium is removable from the layer along the fold line.

13. A container as in claim 1 which includes a flap where the flap closes the receiving region.

14. A container as in claim 13 where the indicium is printed on the flap.

15. A container as in claim 13 where the self-adhesive layer, is at least in part, covered by a removable planar member.

16. A container as in claim 1 where the layers are bonded by one of adhesive, heat sealing, high frequency welding.

17. A container as in claim 16 where the layers are elongated.

18. A container as in claim 16 where one layer is formed of a fibrous material and the other is formed, at least in part of a resin.

19. A container as in claim 18 where the indicium is printed on one of the layers.

20. A container as in claim 18 where the indicium comprises a removable layer.

21. A multi-layer container comprising:
a paper layer;
a plastic layer bonded on three sides to the paper layer;
a closure flap formed on one of the layers;
a sterilization indicator carried by one of the layers, the indicator is at least one of printed on the one layer, or, removably attached to the one layer.

22. A container as in claim 21 where an expansion element is provided along at least some of the sides.

23. A container as in claim 21 which includes a peelable region for separating the layers.

24. A container as in claim 23 where portions of the peelable region are releasably held together.

25. A container as in claim 23 where the paper layer comprises autoclavable paper with a weight parameter in a range of 35-48 pounds.

26. A container as in claim 23 where the plastic layer has a thickness parameter in a range of 1.2 to 2.5 mil.

27. A container as in claim 25 where the plastic layer has a thickness parameter in a range of 1.2 to 2.5 mil.

28. A container as in claim 27 where at least one of the bonds between layers has a central elongated region and first and second regions extending therefrom at a first angle.

29. A container as in claim 28 where another one of the bonds between layers has an elongated section and an end section extending therefrom at a second angle.

30. A container as in claim 29 where one of end regions intersects the end section to form a continuous U-shaped bond between the layers.

31. A container as in claim 29 where the angles are substantially equal.

32. A container as in claim 30 where the peelable region is adjacent to the intersection between the end region and the end section.
